(19) United States
(12) Patent Application Publication
(10) Pub. No.: US 2011/0199500 A1
(43) Pub. Date: Aug. 18, 2011

(54) IMAGE OBTAINING METHOD AND IMAGE CAPTURING APPARATUS

(75) Inventors: Hitoshi SHIMIZU, Saitama-shi (JP); Koji Yoshida, Saitama-shi (JP); Kazuhiko Katakura, Saitama-shi (JP); Takayuki Iida, Saitama-shi (JP)

(73) Assignee: FUJIFILM Corporation, Tokyo (JP)

(21) Appl. No.: 12/975,180

(22) Filed: Dec. 21, 2010

(30) Foreign Application Priority Data
Feb. 18, 2010 (JP) 033534/2010

(51) Int. Cl. H04N 5/228

(52) U.S. Cl. 348/222.1; 348/E07.085

(57) ABSTRACT

Obtaining a first image captured by directing light having a first wavelength to an observation area and receiving light emitted from the observation area, and a second image captured by directing light having a second wavelength shorter than the first wavelength to the observation area and receiving light emitted from the observation area, and obtaining a deep portion image of the observation area by subtracting the second image from the first image.

### BACKGROUND OF THE INVENTION

1. Field of the Invention

The present invention relates to an image obtaining method and an image capturing apparatus for obtaining a deep portion image by directing light having two different wavelengths to obtain two types of images and performing subtraction between the two images.

2. Description of the Related Art

Endoscope systems for observing tissues of body cavities are widely known and an electronic endoscope system that captures an ordinary image of an observation area in a body cavity by directing white light to the observation area and displaying the captured ordinary image on a monitor screen is widely used.

Further, as one of such endoscope systems, a system that obtains a fluorescence image of a blood vessel or a lymphatic vessel by administering, for example, indocyanine green into a body in advance and detecting ICG fluorescence in the blood vessel or lymphatic vessel by directing excitation light to the observation area is known as described, for example, in U.S. Pat. No. 6,804,549 and Japanese Unexamined Patent Publication No. 2007-244746.

Further, U.S. Pat. No. 7,589,839 proposes a method of obtaining a plurality of fluorescence images using a plurality of fluorescent materials.

For example, the blood vessel observation using the ICG described above allows observation of a blood vessel located in a deep layer in the fluorescence image, since near infrared light used as the excitation light has high penetration into a living body. The fluorescence image, however, includes not only the fluorescence image of the blood vessel in the deep layer but also a fluorescence image of a blood vessel in a surface layer, so that the image information of the blood vessel in the surface layer is unnecessary information (artifact) when only the image of the blood vessel in the deep layer is desired to be observed.

The present invention has been developed in view of the circumstances described above, and it is an object of the present invention to provide an image obtaining method and image capturing apparatus capable of obtaining, for example, a deep portion image that allows only an image of blood vessel located in a deep layer to be observed appropriately.

### SUMMARY OF THE INVENTION

An image obtaining method of the present invention is a method including the steps of:
obtaining a first image captured by directing light having a first wavelength to an observation area and receiving light emitted from the observation area, and a second image captured by directing light having a second wavelength shorter than the first wavelength to the observation area and receiving light emitted from the observation area; and
obtaining a deep portion image of the observation area by subtracting the second image from the first image.

An image obtaining method of the present invention is a method including the steps of:
obtaining a first fluorescence image captured by directing excitation light having a first wavelength to an observation area and receiving first fluorescence emitted from the observation area, and a second fluorescence image captured by directing excitation light having a second wavelength shorter than the first wavelength to the observation area and receiving second fluorescence emitted from the observation area; and
obtaining a deep portion fluorescence image of the observation area by subtracting the second fluorescence image from the first fluorescence image.

An image obtaining method of the present invention is a method including the steps of:
obtaining a fluorescence image captured by directing excitation light to an observation area and receiving fluorescence emitted from the observation area, and a narrowband image captured by directing narrowband light having a wavelength shorter than that of the excitation light and a bandwidth narrower than that of white light to the observation area and receiving reflection light reflected from the observation area; and
obtaining a deep portion fluorescence image of the observation area by subtracting the narrowband image from the fluorescence image.

An image capturing apparatus of the present invention is an apparatus including:
a light emission unit for emitting first emission light having a first wavelength and second emission light having a second wavelength shorter than the first wavelength, the first and second emission light being directed to an observation area;
an imaging unit for capturing a first image by receiving light emitted from the observation area irradiated with the first emission light and a second image by receiving light emitted from the observation area irradiated with the second emission light; and
a deep portion image obtaining unit for obtaining a deep portion image of the observation area by subtracting the second image from the first image.

An image capturing apparatus of the present invention is an apparatus including:
a light emission unit for emitting first excitation light having a first wavelength and second excitation light having a second wavelength shorter than the first wavelength, the first and second excitation light being directed to an observation area;
an imaging unit for capturing a first fluorescence image by receiving first fluorescence emitted from the observation area irradiated with the first excitation light and a second fluorescence image by receiving second fluorescence emitted from the observation area irradiated with the second excitation light; and
a deep portion image obtaining unit for obtaining a deep portion image of the observation area by subtracting the second fluorescence image from the first fluorescence image.

In the image capturing apparatus of the present invention described above, near infrared light may be used as the first excitation light.

Further, the light emission unit may be a unit that emits the first excitation light and the second excitation light at the same time, and the imaging unit may be a unit that captures the first fluorescence image and the second fluorescence image at the same time.

An image capturing apparatus of the present invention is an apparatus including:
a light emission unit for emitting excitation light and narrowband light having a wavelength shorter than that of the excitation light and a bandwidth narrower than that of white light, the excitation light and the narrowband light being directed to an observation area;
an imaging unit for capturing a fluorescence image by receiving fluorescence emitted from the observation area irradiated with the excitation light and a narrowband image by receiving reflection light reflected from the observation area irradiated with the narrowband light; and
a deep portion fluorescence image obtaining unit for obtaining a deep portion fluorescence image of the observation area by subtracting the narrowband image from the fluorescence image.

In the image capturing apparatus of the present invention described above, near infrared light may be used as the excitation light.

Further, the light emission unit may be a unit that emits the excitation light and the narrowband light at the same time, and the imaging unit may be a unit that captures the fluorescence image and the narrowband image at the same time.

According to the image obtaining method and image capturing apparatus of the present invention, a first image captured by directing light having a first wavelength to an observation area and receiving light emitted from the observation area, and a second image captured by directing light having a second wavelength shorter than the first wavelength to the observation area and receiving light emitted from the observation area are obtained, and a deep portion image of the observation area is obtained by subtracting the second image from the first image. This allows, for example, subtraction of a second image that includes a blood vessel located only in a surface layer from a first image that includes blood vessels located in the surface layer and a deep layer, whereby a deep portion image that includes a blood vessel located in the deep layer may be obtained.

### BRIEF DESCRIPTION OF THE DRAWINGS

FIG. 1 is an overview of a rigid endoscope system that employs an embodiment of the fluorescence image capturing apparatus of the present invention.

FIG. 2 is a schematic configuration diagram of the body cavity insertion section shown in FIG. 1.

FIG. 3 is a schematic view of a tip portion of a body cavity insertion section according to a first embodiment.

FIG. 4 is a cross-sectional view taken along the line 4-4′ in FIG. 3.

FIG. 5 illustrates a spectrum of light outputted from each light projection unit of the body cavity insertion section according to the first embodiment, and spectra of fluorescence and reflection light emitted/reflected from an observation area irradiated with the light.

FIG. 6 is a schematic configuration diagram of an imaging unit according to a first embodiment.

FIG. 7 illustrates spectral sensitivity of the imaging unit.

FIG. 8 is a block diagram of an image processing unit and a light source unit according to a first embodiment, illustrating schematic configurations thereof.

FIG. 9 is a block diagram of the image processing section shown in FIG. 8, illustrating a schematic configuration thereof.

FIG. 10 is a schematic view illustrating blood vessels of surface and deep layers.

FIG. 11 is a schematic view for explaining a concept of a deep portion fluorescence image generation method.

FIG. 12 is a timing chart illustrating imaging timing of an ordinary image, an ICG fluorescence image and a fluorescein fluorescence image.

FIG. 13 is a flowchart for explaining an operation for displaying an ordinary image, a fluorescence image, and a composite image.

FIG. 14 is a flowchart for explaining line segment extraction using edge detection.

FIG. 15 is a schematic view of a tip portion of a body cavity insertion section according to a second embodiment.

FIG. 16 is a block diagram of an image processing unit and a light source unit according to a second embodiment, illustrating schematic configurations thereof.

FIG. 17 illustrates a spectrum of light outputted from each projection unit of the body cavity insertion section according to the second embodiment, and spectra of fluorescence and reflection light emitted/reflected from an observation area irradiated with the light.

FIG. 18 is a schematic configuration diagram of an imaging unit according to a second embodiment.

FIG. 19 is a schematic view of a tip portion of a body cavity insertion section according to a third embodiment.

FIG. 20 illustrates a spectrum of light outputted from each projection unit of the body cavity insertion section according to the third embodiment, and spectra of fluorescence and reflection light emitted/reflected from an observation area irradiated with the light.

FIG. 21 is a schematic configuration diagram of an imaging unit according to a third embodiment.

### DESCRIPTION OF THE PREFERRED EMBODIMENTS

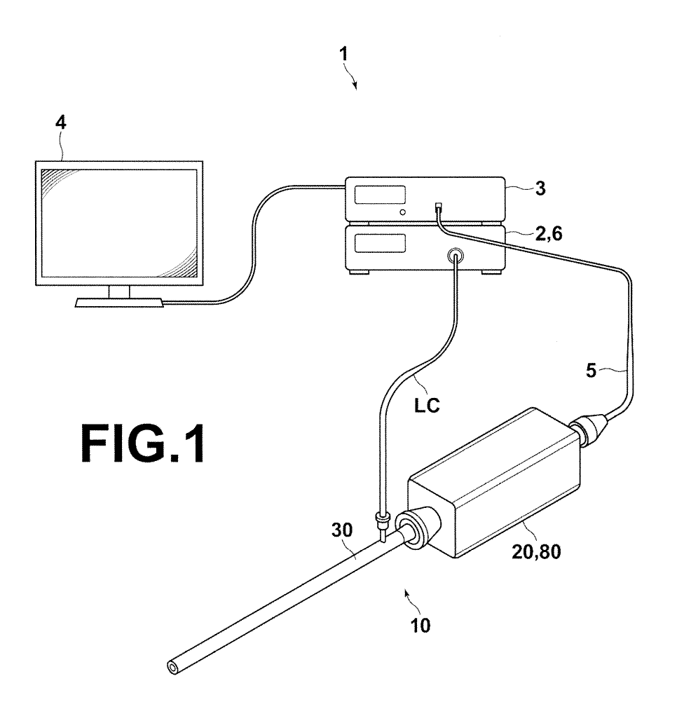

Hereinafter, a rigid endoscope system that employs a first embodiment of the image obtaining method and image capturing apparatus of the present invention will be described with reference to the accompanying drawings. FIG. 1 is an overview of rigid endoscope system 1 of the present embodiment, illustrating a schematic configuration thereof.

As shown in FIG. 1, rigid endoscope system 1 of the present embodiment includes light source unit 2 for emitting two types of excitation light, blue and near infrared light, rigid endoscope imaging unit 10 for guiding and directing the two types of excitation light emitted from light source unit 2 to an observation area and capturing fluorescence images based on fluorescence emitted from the observation area irradiated with the excitation light, image processing unit 3 for performing predetermined processing on image signals obtained by rigid endoscope imaging device 10, and monitor 4 for displaying a deep portion fluorescence image of the observation area based on a display control signal generated in image processing unit 3.

As shown in FIG. 1, rigid endoscope imaging device 10 includes body cavity insertion section 30 to be inserted into a body cavity and imaging unit 20 for capturing an ordinary image and a florescence image of an observation area guided by the body cavity insertion section 30.

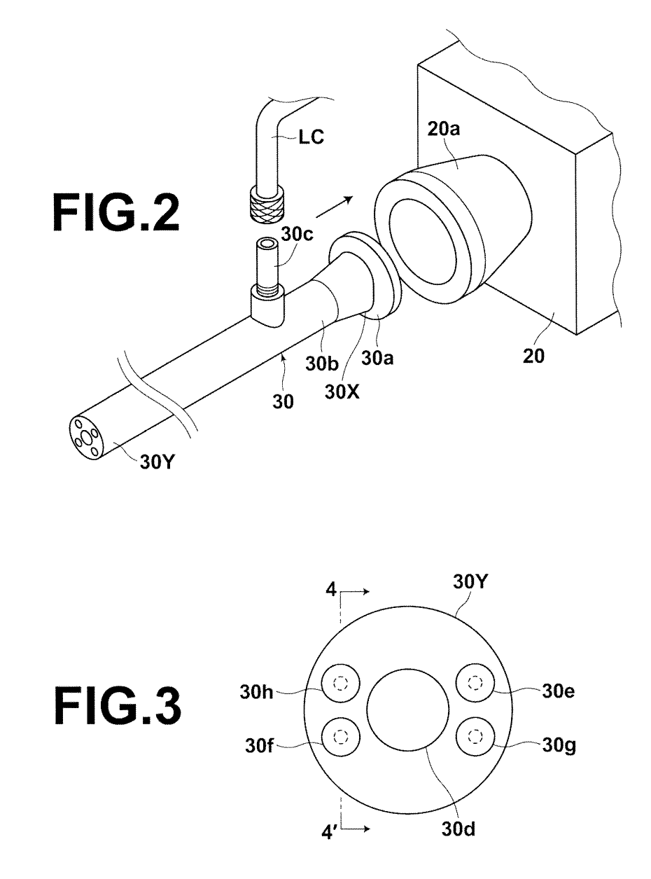

Body cavity insertion section 30 and imaging unit 20 are detachably connected, as shown in FIG. 2. Body cavity insertion section 30 includes connection member 30a, insertion member 30b, and cable connection port 30c.

Connection member 30a is provided at first end 30X of body cavity insertion section 30 (insertion member 30b), and imaging unit 20 and body cavity insertion section 30 are detachably connected by fitting connection member 30a into, for example, aperture 20a formed in imaging unit 20.

Insertion member 30b is a member to be inserted into a body cavity when imaging is performed in the body cavity. Insertion member 30b is formed of a rigid material and has, for example, a cylindrical shape with a diameter of about 5 mm. Insertion member 30b accommodates inside thereof a group of lenses for forming an image of an observation area, and an ordinary image and a fluorescence image of the observation area inputted from second end 30Y are inputted, through the group of lenses, to imaging unit 20 on the side of first end 30X.

Cable connection port 30c is provided on the side surface of insertion member 30b and an optical cable LC is mechanically connected to the port. This causes light source unit 2 and insertion member 30b to be optically connected through the optical cable LC.

As shown in FIG. 3, imaging lens 30d is provided in the approximate center of second end 30Y of body cavity insertion section 30 for forming an ordinary image and a fluorescence image, and white light output lenses 30g and 30h for outputting white light are provide substantially symmetrically across the imaging lens 30d. The reason why two white light output lenses are provide symmetrically with respect to imaging lens 30d is to prevent a shadow from being formed in an ordinary image due to irregularity of the observation area.

Further, blue light output lens 30f for outputting blue light and near infrared light output lens 30e for outputting near infrared light are provided symmetrically with respect to imaging lens 30d at second end 30Y of body cavity insertion section 30.

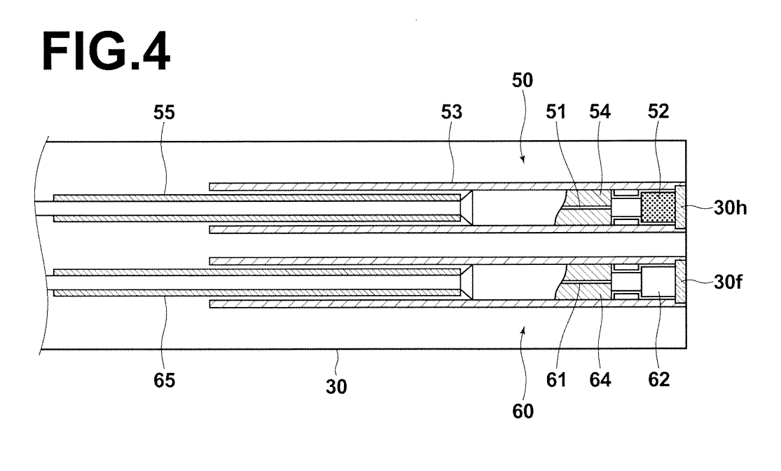

FIG. 4 is a cross-sectional view taken along the line 4-4′ in FIG. 3. As illustrated in FIG. 4, body cavity insertion section 30 includes inside thereof white light projection unit 50 and blue light projection unit 60. White light projection unit 50 includes multimode optical fiber 51 for guiding the blue light and fluorescent body 52 which is excited and emits visible light of green to yellow by absorbing a portion of the blue light guided through multimode optical fiber 51. Fluorescent body 52 is formed of a plurality of types of fluorescent materials, such as a YAG fluorescent material, BAM (BaMgAl₁₀O₁₇), and the like.

Tubular sleeve member 53 is provided so as to cover the periphery of fluorescent body 52, and ferrule 54 for holding multimode optical fiber 51 as the central axis is inserted in sleeve member 53. Further, flexible sleeve 55 is inserted between sleeve member 53 and multimode optical fiber 51 extending from the proximal side (opposite to the distal side) of ferrule 54 to cover the jacket of the fiber.

Blue light projection unit 60 includes multimode optical fiber 61 for guiding the blue light and space 62 is provided between multimode optical fiber 61 and blue light output lens 30f. Also blue light projection unit 60 is provided with tubular sleeve member 63 covering the periphery of space 62, in addition to ferrule 64 and flexible sleeve 65, as in white light projection unit 50.

Then, inside of body cavity insertion section 30, two white light projection units 50 are provided symmetrically with respect to imaging lens 30d, and blue light projection unit 60 and the near infrared light projection unit are provided symmetrically with respect to imaging lens 30d. The near infrared light projection unit has an identical structure to that of the blue light projection unit other than that the near infrared light is guided through the multimode optical fiber. Note that the dotted circle in each output lens in FIG. 3 represents the output end of the multimode optical fiber.

As for the multimode optical fiber used in each light projection unit, for example, a thin optical fiber with a core diameter of 105 μm, a clad diameter of 125 μm, and an overall diameter, including a protective outer jacket, of 0.3 mm to 0.5 mm may be used.

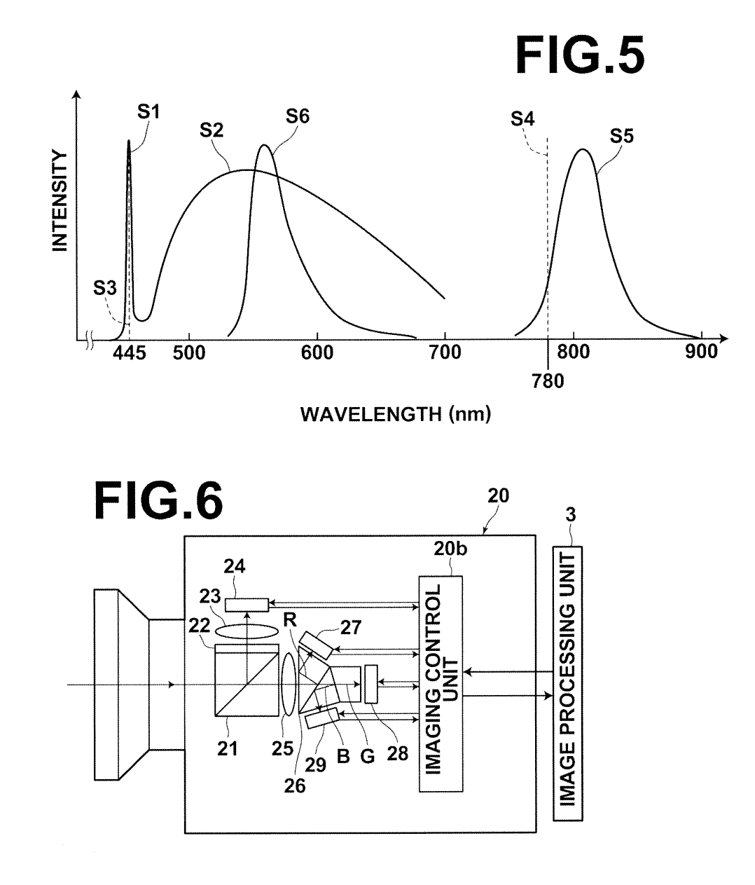

Each spectrum of light outputted from each light projection and spectra of fluorescence and reflection light emitted/reflected from an observation area irradiated with the light outputted from each light source are shown in FIG. 5. FIG. 5 shows a blue light spectrum S1 outputted through fluorescent body 52 of white light projection unit 50, a green to yellow visible light spectrum S2 excited and emitted from fluorescent body 52 of white light projection unit 50, a blue light spectrum S3 outputted from blue light projection unit 60, and a near infrared light spectrum 34 outputted from the near infrared light projection unit.

The term “white light” as used herein is not strictly limited to light having all wavelength components of visible light and may include any light as long as it includes light in a specific wavelength range, for example, primary light of R (red), G (green), or B (blue). Thus, in a broad sense, the white light may include, for example, light having wavelength components from green to red, light having wavelength components from blue to green, and the like. Although white light projection unit 50 emits the blue light spectrum S1 and visible light spectrum S2 shown in FIG. 5, the light of these spectra is also regarded as white light.

FIG. 5 further illustrates an ICG fluorescence spectrum S5 emitted from the observation area irradiated with the near infrared light spectrum S4 outputted from the near infrared light projection unit and a fluorescein fluorescence spectrum S6 emitted from the observation area irradiated with the blue light spectrum S3 outputted from blue light projection unit 60.

FIG. 6 shows a schematic configuration of imaging unit 20. Imaging unit 20 includes a first imaging system for generating a first fluorescence image signal by imaging an ICG fluorescence image emitted from the observation area irradiated with the near infrared excitation light, a second imaging system for generating a second fluorescence image signal by imaging a fluorescein fluorescence image emitted from the observation area irradiated with the blue excitation light, and a third imaging system for generating an ordinary image signal by imaging an ordinary image emitted from the observation area irradiated with the white light.

The first imaging system includes dichroic prism 21 that reflects the ICG fluorescence image emitted from the observation area in a right angle direction, excitation light cut filter 22 that transmits the ICG fluorescence image reflected by dichroic prism 21 and cuts the near infrared excitation light reflected by dichroic prism 21, first image forming system 23 that forms the ICG fluorescence image transmitted through excitation light cut filter 22, and first high sensitivity image sensor 24 that takes the ICG fluorescence image formed by first image forming optical system 23.

The second imaging system includes dichroic prism 21 that transmits the fluorescein fluorescence image emitted from the observation area, second image forming system 25 that forms the fluorescein fluorescence image transmitted through dichroic prism 21, color separation prism 26 that transmits the fluorescein fluorescence image formed by second image forming system 25, and second high sensitivity image sensor 28 that takes the fluorescein fluorescence image transmitted through color separation prism 26.

The third imaging system includes dichroic prism 21 that transmits an ordinary image based on reflection light (visible light) reflected from the observation area irradiated with the white light, second image forming system 25 that forms the ordinary image transmitted through dichroic prism 21, color separation prism 26 that separates the ordinary image formed by second image forming system 25 into R (red), G (green), and B (blue) wavelength ranges, third high sensitivity image sensor 27 that images the red light separated by color separation prism 26, second high sensitivity image sensor 28 that images the green light separated by color separation prism 26, and fourth high sensitivity image sensor 29 that images the blue light separated by color separation prism 26.

Color separation prism 26 doubles as an excitation light cut filter since it separates the blue excitation light on the side of fourth high sensitivity image sensor 29 when the fluorescein fluorescence image is captured.

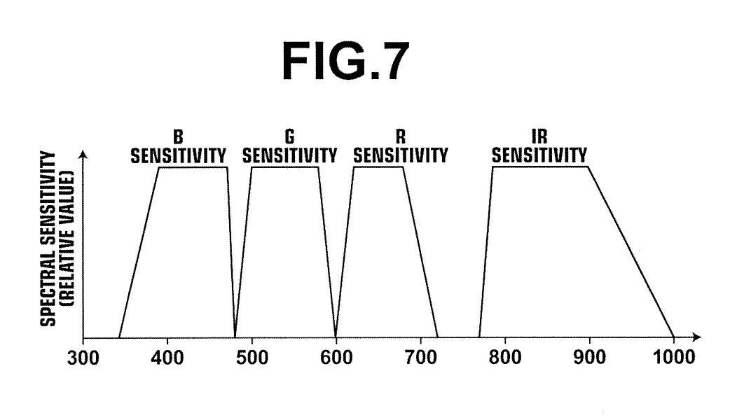

Now, referring to FIG. 7, there is provided a graph of spectral sensitivity of imaging unit 20. More specifically, imaging unit 20 is configured such that the first imaging system has IR (near infrared) sensitivity, the second imaging system has G (green) sensitivity, and the third imaging system has R (red) sensitivity, G (green) sensitivity, and B (blue) sensitivity.

Imaging unit 20 further includes imaging control unit 20b. Imaging control unit 20b is a unit that performs CDS/AGC (correlated double sampling/automatic gain control) and A/D conversion on image signals outputted from high sensitivity image sensors 24 and 27 to 29, and outputs the resultant image signals to image processing unit 3 through cable 5 (FIG. 1).

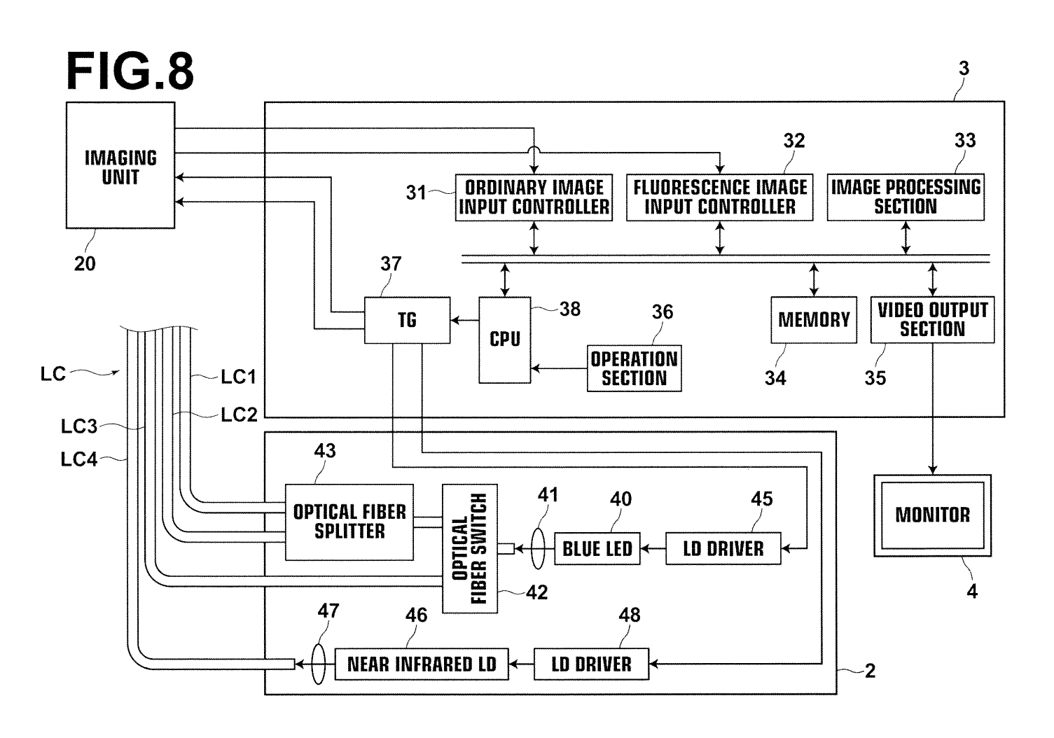

As shown in FIG. 8, image processing unit 3 includes ordinary image input controller 31, fluorescence image input controller 32, image processing section 33, memory 34, video output section 35, operation section 36, TG (timing generator) 37, and CPU 38.

Ordinary image input controller 31 and fluorescence image input controller 32 are each provided with a line buffer having a predetermined capacity and temporarily stores an ordinary image signal formed of image signals of RGB components with respect to one frame, or an ICG fluorescence image signal and an fluorescein fluorescence image signal outputted from imaging control unit 27 of imaging unit 20. Then, the ordinary image signal stored in ordinary image input controller 31 and the fluorescence image signals stored in fluorescence image input controller 32 are stored in memory 34 via the bus.

Image processing section 33 receives the ordinary image signal and fluorescence image signal for one frame read out from memory 34, performs predetermined processing on these image signals, and outputs the resultant image signals to the bus.

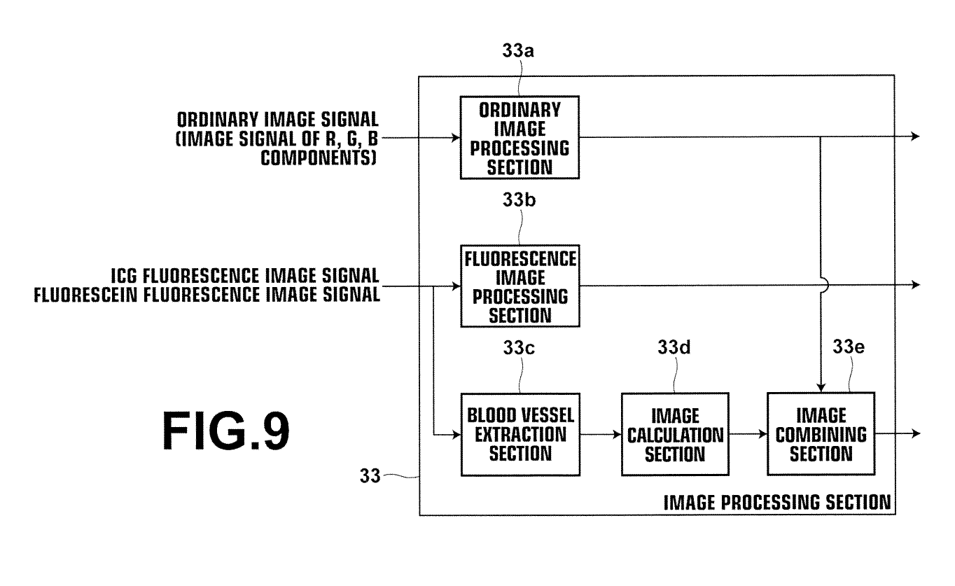

As shown in FIG. 9, image processing section 33 includes ordinary image processing section 33a that performs predetermined image processing, appropriate for an ordinary image, on an inputted ordinary image signal (image signals of RGB components) and outputs the resultant image signal, and fluorescence image processing section 33b that performs predetermined image processing, appropriate for a fluorescence image, on an inputted ICG fluorescence image signal and an fluorescein fluorescence image signal and outputs the resultant image signals, and a blood vessel extraction section that extracts an image signal representing a blood vessel from the ICG fluorescence image signal and fluorescein fluorescence image signal subjected the image processing in fluorescence image processing section 33b. Image processing section 33 further includes image calculation section 33d that subtracts an image signal representing a blood vessel extracted from the fluorescein fluorescence image signal (hereinafter, “fluorescein fluorescence blood vessel image signal”) from an image signal representing a blood vessel extracted from the ICG fluorescence image signal (hereinafter, “ICG fluorescence blood vessel image signal”) and image combining section 33e that generates a deep portion blood vessel image signal based on a result of the calculation of image calculation section 33d and generates a composite image signal by combining the deep portion blood vessel image signal with the ordinary image signal outputted from ordinary image processing section 33a.

Video output section 35 receives the ordinary image signal, fluorescence image signal, and composite image signal outputted from image processing section 33 via the bus, generates a display control signal by performing predetermine processing on the received signals, and outputs the display control signal to monitor 4.

Operation section 36 receives input from the operator, such as various types of operation instructions and control parameters. TG 37 outputs drive pulse signals for driving high sensitivity image sensors 24 and 27 to 29 of imaging unit 20, and LD drivers 45, 48 of light source unit 2, to be described later. CPU 36 performs overall control of the system.

As shown in FIG. 8, light source unit 2 includes blue LD light source 40 that emits 445 nm blue light, condenser lens 41 that condenses the blue light emitted from blue LD light source 40 and inputs the condensed blue light to optical fiber switch 42, optical fiber switch 42 that selectively inputs the received blue light to optical fiber splitter 43 or optical cable LC3, optical fiber splitter 43 that inputs the blue light outputted from optical fiber switch 42 to optical cable LC1 and optical cable LC2 at the same time simultaneously, and LD driver 45 that drives blue LD light source 40.

Light source unit 2 further includes near infrared LD light source 46 that emits 750 to 790 run near infrared light, condenser lens 47 that condenses the near infrared light and inputs the condensed near infrared light to the input end of optical cable LC4, and LD driver 48 that drives near infrared LD light source 46.

In the present embodiment, near infrared light and blue light are used as the two types of excitation light, but excitation light having other wavelengths may also be used as the two types of excitation light as long as the wavelength of either one of them is shorter than that of the other and the excitation light is determined appropriately according to the type of fluorochrome administered to the observation area or the type of living tissue for causing autofluorescence.

Light source 2 is optically coupled to rigid endoscope device 10 through optical cable LC, in which optical cables LC1, LC2 are optically coupled to multimode optical fibers 51 of white light projection unit 50, optical cable LC3 is optically coupled to multimode optical fiber 61 of blue light projection unit 60, and optical cable LC4 is optically coupled to the multimode optical fiber of the near infrared light projection unit.

An operation of the rigid endoscope system of the first embodiment will now be described.

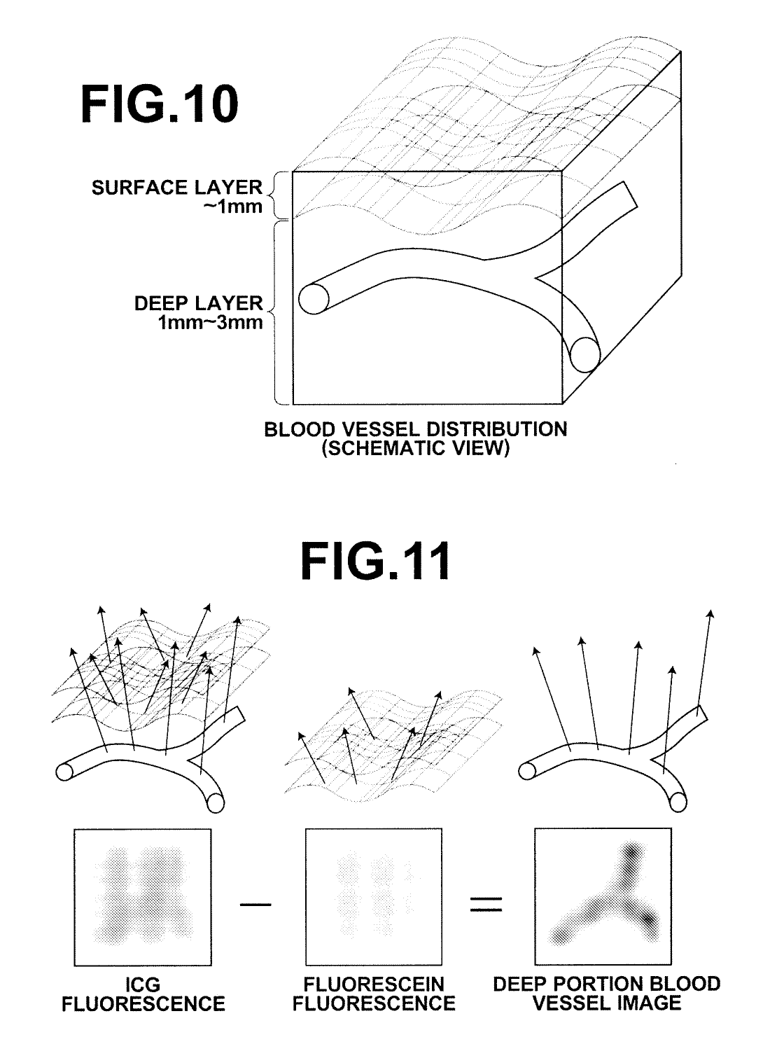

Before going into detailed description of the system operation, the principle of detection of a deep portion blood vessel image to be obtained in the present embodiment will be described using a schematic drawing. In the present embodiment, a deep portion blood vessel located in a deep layer of 1 to 3 mm deep from the body surface is obtained, as shown in FIG. 10. If only an ICG fluorescence image is obtained, the ICG fluorescence image includes not only the deep portion blood vessel image but also image information of a surface layer blood vessel located within a depth of 1 mm from the body surface, so that the surface layer blood vessel image appears as unnecessary information. In the meantime, the excitation light of fluorescein fluorescence is visible light and has low penetration into a living body, so that the fluorescein fluorescence image includes only image information of a surface blood vessel located in a surface layer.

Consequently, in the rigid endoscope system of the present embodiment, a deep portion blood vessel image is obtained by subtracting the fluorescein fluorescence image from the ICG fluorescence image, as illustrated in FIG. 11.

Now, a specific operation of the rigid endoscope system of the present invention will be described.

First, body cavity insertion section 30 with the optical cable LC attached thereto and cable 5 are connected to imaging unit 20 and power is applied to light source unit 2, imaging unit 20, and image processing unit 3 to activate them.

Then, body cavity insertion section 30 is inserted into a body cavity by the operator and the tip of body cavity insertion section 30 is placed adjacent to an observation area. Here, it is assumed that ICG and fluorescein have already been administered to the observation area.

Here, an operation of the system for capturing an ICG fluorescence image and an ordinary image will be described first. When capturing an ICG fluorescence image and an ordinary image, blue light emitted from blue LD light source 40 of light source unit 2 is inputted, among optical cables LC1 to LC2, only to LC1 and LC2 through condenser lens 41, optical fiber switch 42, and optical fiber splitter 43. Then, the blue light is guided through optical cables LC1 and LC2 and inputted to body cavity insertion section 30, and further guided through multimode optical fibers 51 of white light projection unit 50 in body cavity insertion section 30. Thereafter, a portion of the blue light outputted from the output end of each multimode optical fiber 51 is transmitted through fluorescent body 52 and directed to the observation area, while the remaining blue light other than the portion is subjected to wavelength conversion to green to yellow visible light by fluorescent body 52 and directed to the observation area. That is, the observation area is irradiated with white light formed of the blue light and green to yellow visible light.

In the mean time, near infrared light emitted from near infrared LD light source 46 of light source unit 2 is inputted to body cavity insertion section 30 through condenser lens 47 and optical cable LC4. Then, the near infrared light is guided through the multimode optical fiber of the near infrared light projection unit in body cavity insertion section 30 and directed to the observation area simultaneously with the white light.

Then, an ordinary image based on reflection light reflected from the observation area irradiated with the white light and an ICG fluorescence image based on ICG fluorescence emitted from the observation area irradiated with the near infrared light are captured simultaneously.

More specifically, an ordinary image is captured in the following manner. Reflection light reflected from the observation area irradiated with the white light is inputted to insertion member 30b from imaging lens 30d at the tip 30Y of insertion member 30b, then guided by the group of lenses inside of the insertion member 30b, and outputted to imaging unit 20.

The reflection light inputted to imaging unit 20 is transmitted through dichroic prism 21 and second image forming system 25, then separated into R, G, and B wavelength ranges by color separation prism 26, and the red light is imaged by third high sensitivity image sensor 27, the green light is imaged by second high sensitivity image sensor 28, and the blue light is imaged by fourth high sensitivity image sensor 29.

Then, R, G, and B image signals outputted from second to fourth high sensitivity image sensors 27 to 29 respectively are subjected to CDS/AGC (correlated double sampling/automatic gain control) and A/D conversion in imaging control unit 27, and outputted to image processing unit 3 through cable 5.

In the mean time, the ICG fluorescence image is captured in the following manner. The ICG fluorescence image emitted from the observation area irradiated with the blue excitation light is inputted to insertion member 30b from imaging lens 30d at the tip 30Y of insertion member 30b, then guided by the group of lenses inside of the insertion member 30b, and outputted to imaging unit 20.

The ICG fluorescence image inputted to imaging unit 20 is reflected in a right angle direction by dichroic prism 21, then passed through excitation light cut filter 22, formed on the imaging surface of first high sensitivity image sensor 24 by first image forming system 23, and imaged by first high sensitivity image sensor 24. The ICG fluorescence image signal outputted from first high sensitivity image sensor 24 is subjected to CDS/AGC (correlated double sampling/automatic gain control) and A/D conversion in imaging control unit 27, and outputted to image processing unit 3 through cable 5.

Next, an operation of the system for capturing a fluorescein fluorescent image will be described.

A fluorescein fluorescent image is captured in the following manner. When capturing a fluorescein fluorescent image, blue light emitted from blue LD light source 40 of light source unit 2 is inputted, among optical cables LC1 to LC2, only to LC3 through condenser lens 41 and optical fiber switch 42. Then, the blue light is guided through optical cable LC3 and inputted to body cavity insertion section 30, and further guided through multimode optical fiber 61 of blue light projection unit 60 in body cavity insertion section 30. Thereafter, the blue light outputted from the output end of multimode optical fiber 61 is passed through space 62 and directed to the observation area.

Then, a fluorescein fluorescent image emitted from the observation area irradiated with the blue light is inputted to insertion member 30b from imaging lens 30d at the tip 30Y of insertion member 30b, then guided by the group of lenses inside of the insertion member 30b, and outputted to imaging unit 20.

The fluorescein fluorescent image inputted to imaging unit 20 is transmitted through dichroic prism 21, second image forming system 25, and color separation prism 26, and imaged by second high sensitivity image sensor 28.

The Fluorescein fluorescence image signal outputted from second high sensitivity image sensor 28 is subjected to CDS/AGC (correlated double sampling/automatic gain control) and A/D conversion in imaging control unit 27, and outputted to image processing unit 3 through cable 5.

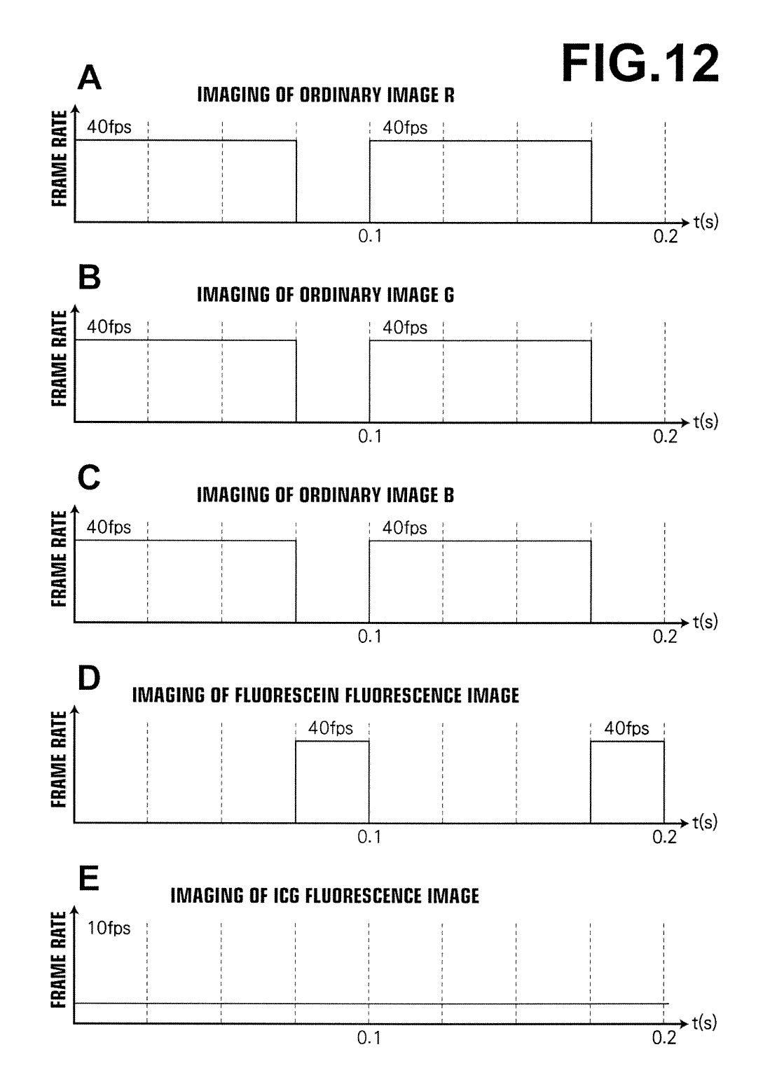

Now, referring to A to E of FIG. 12, there is provided timing charts illustrating imaging timing of each of the ordinary image, ICG image, and fluorescein fluorescence image described above. In each of the timing charts A to E of FIG. 12, the horizontal axis represents elapsed time and vertical axis represents frame rate of the high sensitivity image sensor.

A of FIG. 12 shows the imaging timing of third high sensitivity image sensor 27 for imaging R image signal, B of FIG. 12 shows the imaging timing of second high sensitivity image sensor 28 for imaging G image signal, C of FIG. 12 shows the imaging timing of fourth high sensitivity image sensor 29 for imaging B image signal, D of FIG. 12 shows the imaging timing of second high sensitivity image sensor 28 for imaging fluorescein fluorescence image signal, and E of FIG. 12 shows the imaging timing of first high sensitivity image sensor 24 for imaging ICG fluorescence image signal.

In the timing charts of R, G, and B image signals shown in A to C of FIG. 12, the imaging is performed with a period of 0.1 sec, a duty ratio of 0.75, and a frame rate of 40 fps. In the timing chart of fluorescein fluorescence image signal shown in D of FIG. 12, the imaging is performed with a period of 0.1 sec, a duty ratio of 0.25, and a frame rate of 40 fps. In the timing chart of ICG fluorescence image signal shown in E of FIG. 12, the imaging is performed with a duty ratio of 1 and a frame rate of 10 fps.

As the ordinary image and fluorescein fluorescence image have the same G color component and can not be imaged at the same time, they are imaged at different timing as shown in A to C and D of FIG. 12.

Note that blue LD light source 40 and near infrared LD light source 46 in light source unit 2 are drive controlled according to the timing charts of A to E of FIG. 12.

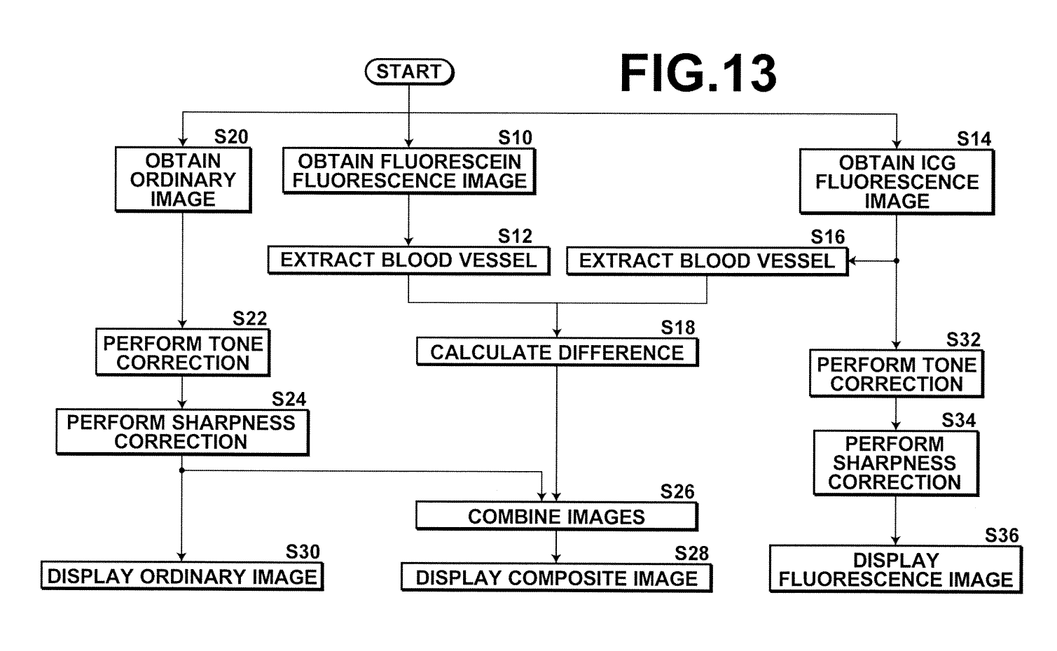

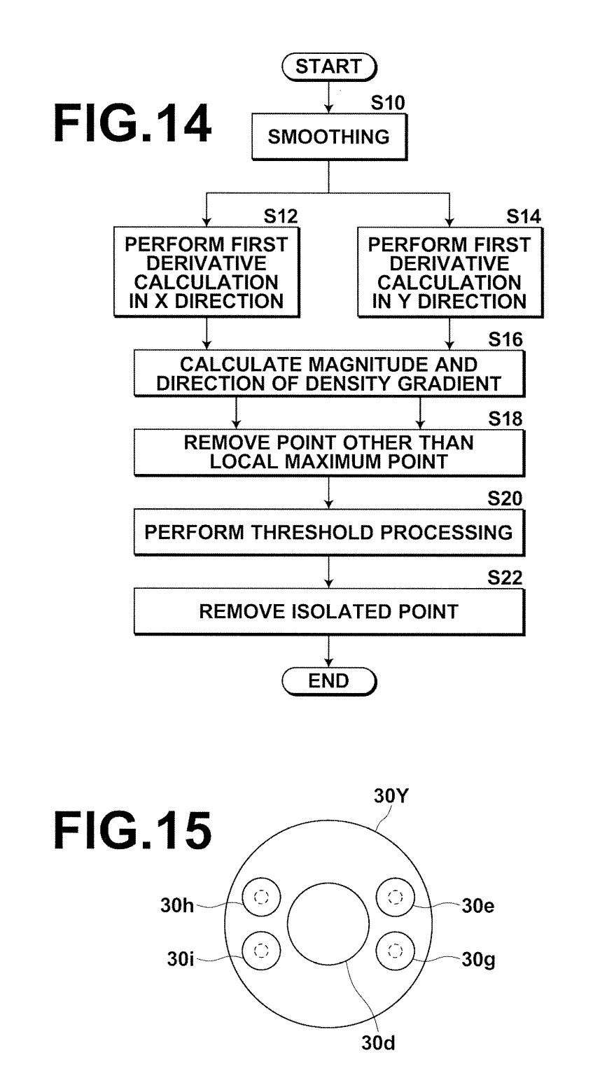

Next, an operation of the system for displaying an ordinary image, a fluorescence image, and a composite image based on the ordinary image signal formed of R, G, and B image signals, ICG fluorescence image signal, and fluorescein fluorescence image signal obtained by imaging unit 20 will be described with reference to FIGS. 8, 9, and flowcharts shown in FIGS. 13, 14.

An operation for displaying the ordinary image and ICG fluorescence image will be described first. The ordinary image signal formed of R, G, and B image signals inputted to image processing unit 3 is temporarily stored in ordinary image input controller 31 and then stored in memory 34 (FIG. 13, S20). Ordinary image signals for one frame read out from memory 34 are subjected to tone correction and sharpness correction in ordinary image processing section 33a of image processing section 33 (FIG. 13, S22, S24), and outputted to video output section 35.

Video output section 35 generates a display control signal by performing predetermined processing on the inputted ordinary image signal and outputs the display control signal to monitor 4. Monitor 4, in turn, displays an ordinary image based on the inputted display control signal (FIG. 13, S30).

The ICG fluorescence image signal inputted to image processing unit 3 is temporarily stored in fluorescence image input controller 32 and then stored in memory 34 (FIG. 13, S14). ICG fluorescence image signals for one frame read out from memory 34 are subjected to tone correction and sharpness correction in fluorescence image processing section 33b of image processing section 33 (FIG. 13, S32, S34), and outputted to video output section 35.

Video output section 35 generates a display control signal by performing predetermined processing on the inputted ICG fluorescence image signal and outputs the display control signal to monitor 4. Monitor 4, in turn, displays an ICG fluorescence image based on the inputted display control signal (FIG. 13, S36).

Next, an operation of the system for generating a deep portion blood vessel image based on the ICG fluorescence image signal and fluorescein fluorescence image, and displaying a composite image combining the deep portion blood vessel image and ordinary image will be described.

The fluorescein fluorescence image signal inputted to image processing unit 3 is temporarily stored in fluorescence image input controller 32 and then stored in memory 34 (FIG. 13, S10).

Then, the fluorescein fluorescence image signal and ICG fluorescence image signal stored in memory 34 are inputted to blood vessel extraction unit 33c of image processing section 33. Then, in blood vessel extraction unit 33c, blood vessel extraction processing is performed on each image signal (FIG. 13, S12, S16).

The blood vessel extraction may be implemented by performing line segment extraction. In the present embodiment, the line segment extraction is implemented by performing edge detection and removing an isolated point from the edge detected by the edge detection. Edge detection methods include, for example, Canny method using first derivation. A flowchart for explaining the line segment extraction using the Canny edge detection is shown in FIG. 14.

As shown in FIG. 14, filtering using a DOG (derivative of Gaussian) filter is performed on each of the ICG fluorescence image signal and fluorescein fluorescence image signal (FIGS. 14, S10 to 514). The filtering using the DOG filter is combined processing of Gaussian filtering (smoothing) for noise reduction with first derivative filtering in x, y directions for density gradient detection.

Thereafter, with respect to each of ICG fluorescence image signal and fluorescein fluorescence image signal subjected to the filtering, the magnitude and direction of the density gradient are calculated (FIG. 14, S16). Then, a local maximum point is extracted and non-maxima other than the local maximum point are removed (FIG. 14, S18).

Then, the local maximum point is compared to a predetermined threshold value and a local maximum point with a value greater than or equal to the threshold value is detected as an edge (FIG. 14, S20). Further, an isolated point which is a local maximum point having a value greater than or equal to the threshold value but does not form a continuous edge is removed (FIG. 14, S22). The removal of the isolated point is processing for removing an isolated point not suitable as an edge from the detection result. More specifically, the isolated point is detected by checking the length of each detected edge.

The edge detection algorithm is not limited to that described above and the edge detection may also be performed using a LOG (Laplace of Gaussian) filter that combines Gaussian filtering for noise reduction with a Laplacian filter for edge extraction through secondary differentiation.

In the present embodiment, a blood vessel is extracted by line segment extraction using edge detection, but the method of blood vessel extraction is not limited to this and any method may be employed as long as it is designed for extracting a blood vessel portion, such as a method using hue or luminance.

With respect to each of the ICG fluorescence image signal and fluorescein fluorescence image signal, an ICG fluorescence blood vessel image signal and a fluorescein fluorescence blood vessel image signal are generated by extracting a blood vessel in the manner as described above. The fluorescein fluorescence blood vessel image signal represents an image of a surface layer blood vessel located in a surface layer from the body surface of the observation area to a depth of 1 mm, while the ICG fluorescence blood vessel image signal includes both the surface layer blood vessel and a deep portion blood vessel located in a deep layer of a depth of 1 to 3 mm from the body surface.

Then, the ICG fluorescence blood vessel image signal and fluorescein fluorescence blood vessel image signal generated in blood vessel extraction section 33c are outputted to image calculation section 33d where a deep portion blood vessel image is generated based on these signals. More specifically, the deep portion blood vessel image is generated by subtracting the fluorescein fluorescence image signal from the ICG fluorescence image signal (FIG. 13, S18).

The deep portion blood vessel image generated in image calculation section 33d in the manner as described above is outputted to image combining section 33e. Image combining section 33e also receives the ordinary image signal outputted from ordinary image processing section 33a, and combines the ordinary image signal and deep portion blood vessel image signal to generate a composite image signal (FIG. 13, S26)

The composite image signal generated in image combining section 33e is outputted to video output section 35. Video output section 35 generates a display control signal by performing predetermine processing on the inputted composite image signal, and outputs the display control signal to monitor 4. Monitor 4 displays a composite image based on the inputted display control signal (FIG. 13, S28).

Next, a rigid endoscope system that employs a second embodiment of the image obtaining method and image capturing apparatus of the present invention will be described in detail. In the rigid endoscope system of the second embodiment obtains a narrowband image using green narrowband light instead of the fluorescein fluorescence image obtained in the rigid endoscope system of the first embodiment.

The overall configuration of the rigid endoscope system of the second embodiment is identical to that of the rigid endoscope system of the first embodiment shown in FIG. 1. Hereinafter, the description will be made focusing on the configuration different from that of the rigid endoscope system of the first embodiment.

Referring to FIG. 15, there is provided a configuration of tip portion 30Y of body cavity insertion section 30 of the rigid endoscope system of the present embodiment. As shown in FIG. 15, a green light output lens 30i for outputting green narrowband light is provided in the present embodiment instead of blue light output lens 30f in the first embodiment. Further, a green light projection unit is provided instead of blue light projection unit 60, but the configuration thereof is identical to that of blue light projection unit 60 illustrated in FIG. 4 and, therefore, will not be elaborated upon further here.

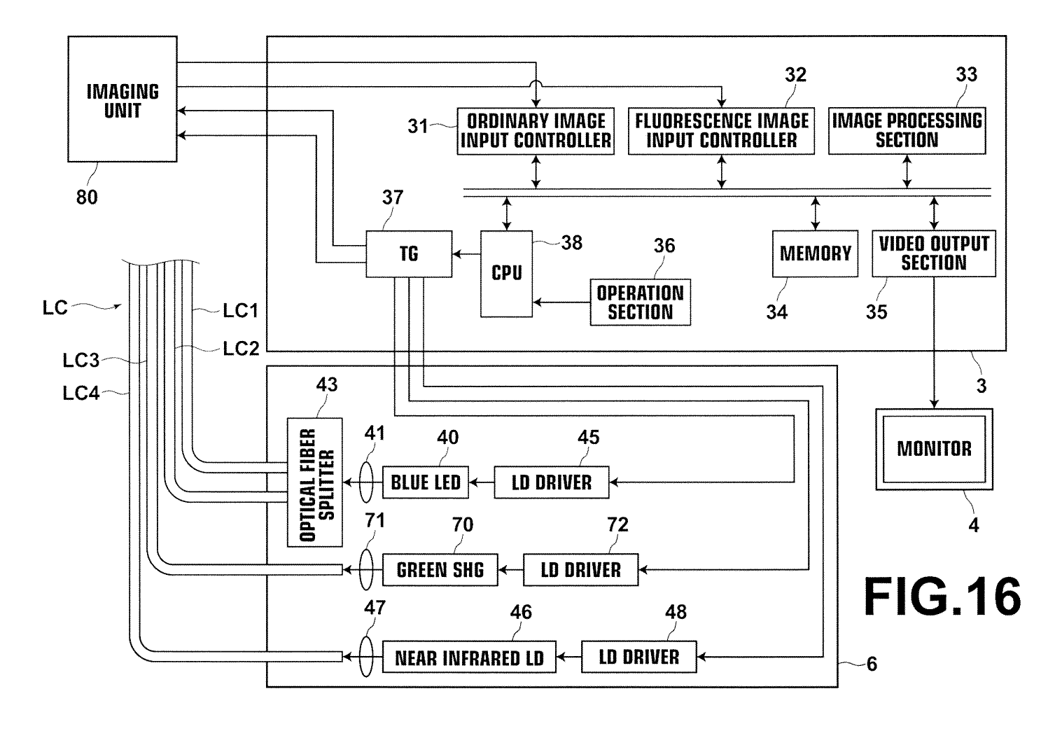

Referring to FIG. 16, there is provided a configuration of light source unit 6 of the rigid endoscope system of the present invention. In comparison with light source unit 2 according to the first embodiment, light source unit 6 further includes green wavelength conversion laser light source 70, condenser lens 71 that condenses the green light emitted from green wavelength conversion laser light source 70 and inputs the condensed green light to the input end of optical fiber LC3, and LD driver 72 that drives green wavelength conversion laser light source 70, as illustrated in FIG. 16. Light source unit 6 of the present embodiment does not include optical fiber switch 42, but other configurations are identical to those of light source unit 2.

Light source 6 is optically coupled to rigid endoscope device 10 through optical cable LC, in which optical cables LC1, LC2 are optically coupled to multimode optical fibers 51 of white light projection unit 50, optical cable LC3 is optically coupled to the multimode optical fiber of the green light projection unit, and optical cable LC4 is optically coupled to the multimode optical fiber of the near infrared light projection unit.

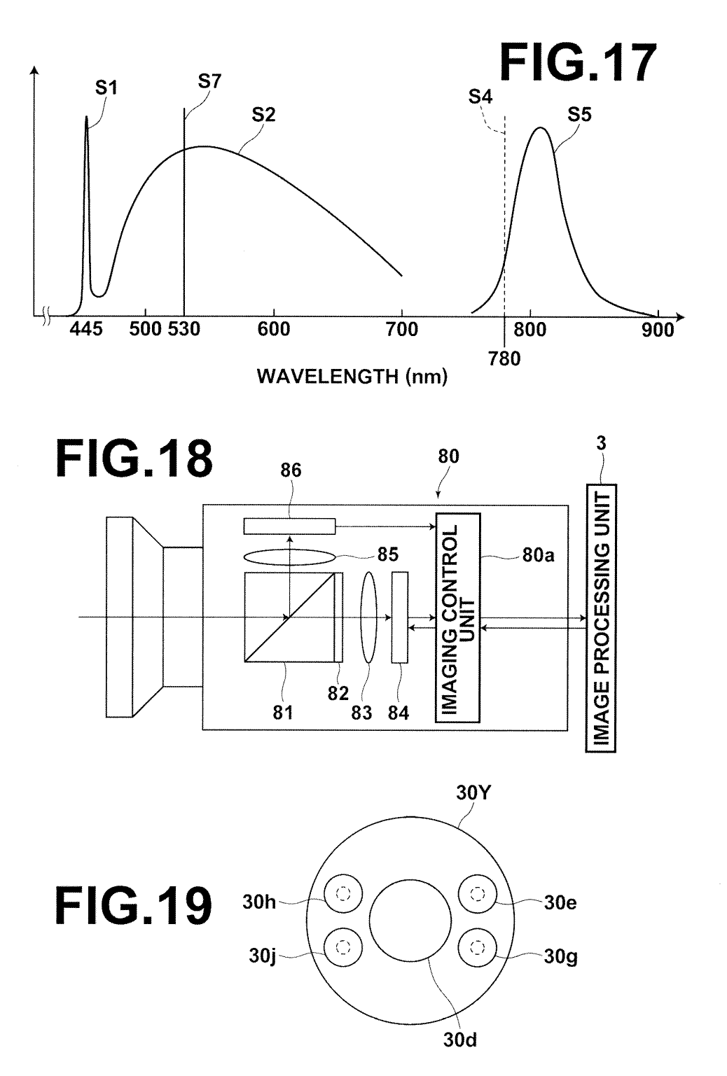

Each Spectrum of light outputted from each light projection unit provided inside of body cavity insertion section 30 of the present embodiment and spectra of fluorescence and reflection light emitted/reflected from an observation area irradiated with the light outputted from each light projection unit are shown in FIG. 17. FIG. 17 shows a blue light spectrum Si outputted through fluorescent body 52 of white light projection unit 50, a green to yellow visible light spectrum S2 excited and emitted from fluorescent body 52 of white light projection unit 50, a green light spectrum S7 outputted from the green light projection unit, and a near infrared light spectrum S4 outputted from near infrared projection unit.

FIG. 17 further illustrates an ICG fluorescence spectrum S5 emitted from the observation area irradiated with the near infrared light spectrum S4 outputted from the near infrared light projection unit. Note that the spectrum S7 of green light outputted from the green light projection unit and a spectrum of the reflection light thereof are identical.

The green light outputted from the green light projection unit has a wavelength of 530 nm to 550 nm which is shorter than that of the near infrared light and is narrowband light with a bandwidth of 20 nm which is narrower than that of the white light. In the present embodiment, the green light is used, but light in other wavelength ranges may be used as long as it has a shorter wavelength than that of the near infrared excitation light and a narrower bandwidth than that of the white light.

Now referring to FIG. 18, there is provided a schematic configuration of imaging unit 80 of the present embodiment. Imaging unit 80 includes a first imaging system for generating an ICG fluorescence image signal of an observation area by imaging ICG fluorescence emitted from the observation area irradiated with the near infrared excitation light and a second imaging system for generating a green narrowband image signal by capturing a green narrowband image reflected from the observation area irradiated with the green narrowband light and an ordinary image signal of the observation area by capturing an ordinary image reflected from the observation area irradiated with the white light.

The first imaging system includes dichroic prism 81 that transmits the ICG fluorescence image emitted from the observation area, excitation light cut filter 82 that transmits the ICG fluorescence image transmitted through dichroic prism 81 and cuts the near infrared excitation light transmitted through dichroic prism 81, first image forming system 83 that forms the ICG image transmitted through excitation light cut filter 82, and first high sensitivity image sensor 84 that takes the ICG fluorescence image formed by first image forming system 83.

The second imaging system includes dichroic prism 81 that reflects the ordinary image and green narrowband image reflected from the observation area in a right angle direction, second image forming system 85 that forms the ordinary image and green narrowband image reflected by dichroic mirror, and second high sensitivity image sensor 86 that takes the ordinary image and green narrowband image formed by second image forming system 85 at different timing. Color filters of three primary colors, red (R), green (G), and blue (B) are arranged on the imaging surface of second high sensitivity image sensor 66 in a Beyer or honeycomb pattern.

The spectral sensitivity of imaging unit 80 is identical to that of the first embodiment illustrated in FIG. 7.

Imaging unit 80 further includes imaging control unit 80a. Imaging control unit 80a is a unit that performs CDS/AGC (correlated double sampling/automatic gain control) and A/D conversion on image signals outputted from first and second high sensitivity image sensors 84, 86 and outputs the resultant image signals to image processing unit 3 through cable 5 (FIG. 1).

The configuration of image processing unit is identical to that of rigid endoscope system of the first embodiment.

An operation of the rigid endoscope system of the second embodiment will now be described.

As described above, the rigid endoscope system of the present embodiment obtains the green narrowband image instead of the fluorescein fluorescence image obtained in the rigid endoscope system of the first embodiment and a deep portion blood vessel image is obtained by subtracting the green narrowband image from the ICG fluorescence image.

Hereinafter, a specific operation of the rigid endoscope system of the present embodiment will be described.

First, an operation of the system for capturing an ICG fluorescence image and an ordinary image will be described. When capturing an ICG fluorescence image and an ordinary image, blue light emitted from blue LD light source 40 of light source unit 6 is inputted to optical cables LC1, LC2 through condenser lens 41 and optical fiber splitter 43. Then, the blue light is guided through optical cables LC1 and LC2 and inputted to body cavity insertion section 30, and further guided through multimode optical fibers 51 of white light projection unit 50 in body cavity insertion section 30. Thereafter, a portion of the blue light outputted from the output end of each multimode optical fiber 51 is transmitted through fluorescent body 52 and directed to the observation area, while the remaining blue light other than the portion is subjected to wavelength conversion to green to yellow visible light by fluorescent body 52 and directed to the observation area. That is, the observation area is irradiated with white light formed of the blue light and green to yellow visible light.

In the mean time, near infrared light emitted from near infrared LID light source 46 of light source unit 6 is inputted to body cavity insertion section 30 through condenser lens 47 and optical cable LC4. Then, the near infrared light is guided through the multimode optical fiber of the near infrared light projection unit in body cavity insertion section 30 and directed to the observation area simultaneously with the white light.

Then, an ordinary image based on reflection light reflected from the observation area irradiated with the white light and an ICG fluorescence image based on ICG fluorescence emitted from the observation area irradiated with the near infrared light are captured simultaneously.

More specifically, an ordinary image is captured in the following manner. Reflection light reflected from the observation area irradiated with the white light is inputted to insertion member 30b from imaging lens 30d at the tip 30Y of insertion member 30b, then guided by the group of lenses inside of the insertion member 30b, and outputted to imaging unit 80.

The ordinary image inputted to imaging unit 80 is reflected by dichroic prism 81 in a right angle direction and formed on the imaging surface of second high sensitivity image sensor 86 by second image forming system 85 and imaged by second high sensitivity image sensor 86.

The R, G, B image signals outputted from second high sensitivity image sensor 86 are subjected to CDS/AGC (correlated double sampling/automatic gain control) and A/D conversion in imaging control unit 80a, and outputted to image processing unit 3 through cable 5.

In the mean time, the ICG fluorescence image is captured in the following manner. The ICG fluorescence image emitted from the observation area irradiated with the blue excitation light is inputted to insertion member 30b from imaging lens 30d at the tip 30Y of insertion member 30b, then guided by the group of lenses inside of the insertion member 30b, and outputted to imaging unit 80.

The ICG fluorescence image inputted to imaging unit 80 is transmitted through dichroic prism 81 and excitation light cut filter 82, and formed on the imaging plane of first high sensitivity image sensor 84 by first image forming system 83 and imaged by first high sensitivity image sensor 84. The ICG fluorescence image signal outputted from first high sensitivity image sensor 84 is subjected to CDS/AGC (correlated double sampling/automatic gain control) and A/D conversion in imaging control unit 80a, and outputted to image processing unit 3 through cable 5.

Next, an operation of the system for capturing a green narrowband image will be described. When capturing a green narrowband image, green narrowband light emitted from green wavelength conversion laser light source 70 of light source unit 6 is inputted to optical cable LC3 through condenser lens 71. Then, the green narrowband light is guided through optical cable LC3 and inputted to body cavity insertion section 30, and further guided through the multimode optical fiber of the green light projection unit in body cavity insertion section 30. Then, the green narrowband light is outputted from the output end of the multimode optical fiber and directed to the observation area.

A green narrowband image reflected from the observation area irradiated with the green narrowband light is inputted to insertion member 30b from imaging lens 30d at the tip 30Y of insertion member 30b, then guided by the group of lenses inside of the insertion member 30b, and outputted to imaging unit 80.

The green narrowband image inputted to imaging unit 80 is reflected in a right angle direction by dichroic prism 81, then formed on the imaging surface of second high sensitivity image sensor 86 by second image forming system 85, and imaged by second high sensitivity image sensor 86 through the green (G) filters on the imaging surface thereof.

The green narrowband image signal outputted from second sensitivity image sensor 86 are subjected to CDS/AGC (correlated double sampling/automatic gain control) and A/D conversion in imaging control unit 80a, and outputted to image processing unit 3 through cable 5.

Note that the imaging timing of the ordinary image, ICG fluorescence image, and green narrowband image is identical to that of A to C, E, and D of FIG. 12 respectively.

Also note that blue LD light source 40 and near infrared LD light source 46 in light source unit 6 are drive controlled according to the timing charts of A to E of FIG. 12.

Then, an ordinary image, an ICG fluorescence image, and a composite image are displayed based on the ordinary image signal formed of the R, G, and B signals, ICG fluorescence image signal, and green narrowband fluorescence image signal obtained by imaging unit 80 in the manner as described above. The operation of the system for displaying these images is identical to that of the rigid endoscope system of the first embodiment shown in the flowcharts of FIG. 13, 14 except that the green narrowband fluorescence image signal is used instead of the fluorescein fluorescence image signal. Therefore, the operation will not be elaborated upon further here.

Next, a rigid endoscope system that employs a third embodiment of the image obtaining method and image capturing apparatus of the present invention will be described in detail. In the rigid endoscope system of the third embodiment obtains a luciferase fluorescence image using ultraviolet light instead of the fluorescein fluorescence image obtained in the rigid endoscope system of the first embodiment.

The overall configuration of the rigid endoscope system of the third embodiment is identical to that of the rigid endoscope system of the first embodiment shown in FIG. 1. Hereinafter, the description will be made focusing on the configuration different from that of the rigid endoscope system of the first embodiment.

Referring to FIG. 19, there is provided a configuration of tip portion 30Y of body cavity insertion section 30 of the rigid endoscope system of the present embodiment. As shown in FIG. 19, an ultraviolet light output lens 30j for outputting ultraviolet light is provided in the present embodiment instead of blue light output lens 30f in the first embodiment. Further, an ultraviolet light projection unit is provided instead of blue light projection unit 60, but the configuration thereof is identical to that of blue light projection unit 60 illustrated in FIG. 4 and, therefore, will not be elaborated upon further here.

The light source unit of the rigid endoscope system of the present embodiment is identical to light source unit 6 of the second embodiment except that an ultraviolet light source is provided instead of green wavelength conversion laser light source 70.

Ultraviolet light emitted from the ultraviolet laser light source of the present embodiment is inputted to optical cable LC3, guided through optical cable LC3, and inputted to the multimode optical fiber of the ultraviolet light projection unit.

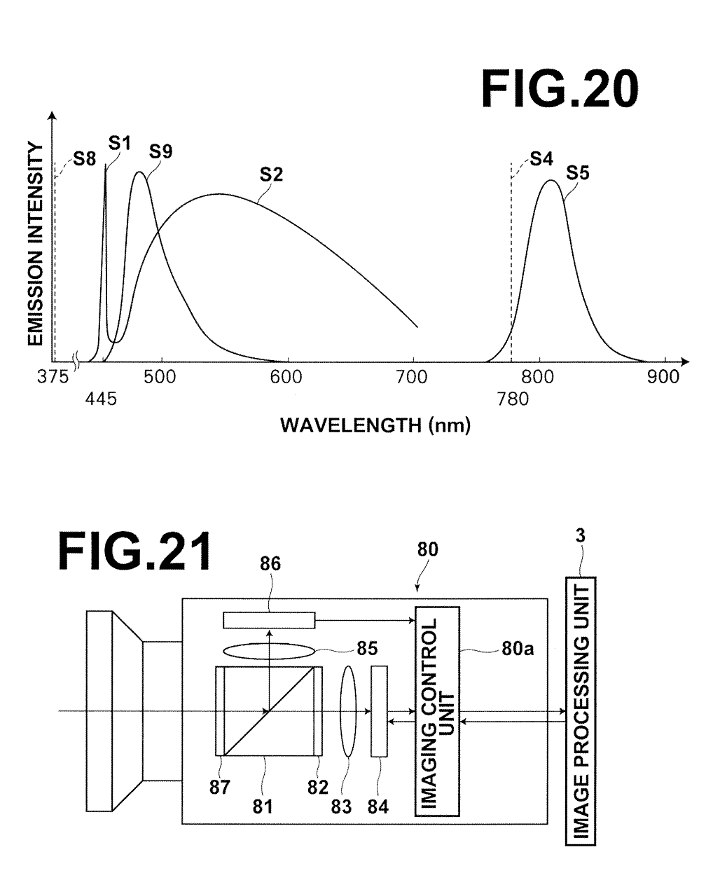

Each Spectrum of light outputted from each light projection unit provided inside of body cavity insertion section 30 of the present embodiment and spectra of fluorescence and reflection light emitted/reflected from an observation area irradiated with the light outputted from each light projection unit are shown in FIG. 20. FIG. 20 shows a blue light spectrum Si outputted through fluorescent body 52 of white light projection unit 50, a green to yellow visible light spectrum S2 excited and emitted from fluorescent body 52 of white light projection unit 50, an ultraviolet light spectrum S8 outputted from the ultraviolet light projection unit, and a near infrared light spectrum S4 outputted from near infrared projection unit.

FIG. 20 further illustrates an ICG fluorescence spectrum S5 emitted from the observation area irradiated with the near infrared light spectrum S4 outputted from the near infrared light projection unit and luciferase fluorescence spectrum S9 emitted from the observation area irradiated with the ultraviolet light spectrum S8 outputted from the ultraviolet light projection unit.

As shown in FIG. 20, the ultraviolet light outputted from the ultraviolet light projection unit is light having a wavelength of around 375 nm which is shorter than that of the near infrared light.

Now referring to FIG. 21, there is provided a schematic configuration of imaging unit 80 of the present embodiment. Imaging unit 80 includes a first imaging system for generating an ICG fluorescence image signal of an observation area by imaging ICG fluorescence emitted from the observation area irradiated with the near infrared excitation light and a second imaging system for generating a luciferase fluorescence image signal by capturing a luciferase image reflected from the observation area irradiated with the ultraviolet light and an ordinary image signal of the observation area by capturing an ordinary image reflected from the observation area irradiated with the white light.

Imaging unit 80 of the present embodiment is identical to imaging unit 80 of second embodiment except that it further includes ultraviolet light cut filter 87 for cutting ultraviolet light. Ultraviolet light cut filter 87 is formed of a high-pass filter for cutting the ultraviolet wavelength range of 375 nm and is provided at the light incident surface of dichroic prism 81. Other configurations are identical to those of imaging unit 80 of the second embodiment described above.

Further, the configuration of image processing unit 3 is identical to that of the rigid endoscope system of the first or second embodiment.

An operation of the rigid endoscope system of the third embodiment will now be described.

As described above, the rigid endoscope system of the present embodiment obtains a luciferase fluorescence image instead of the fluorescein fluorescence image obtained in the rigid endoscope system of the first embodiment and a deep portion blood vessel image is obtained by subtracting the luciferase fluorescence image from the ICG fluorescence image.

The operation of the system of the present embodiment for imaging the ICG fluorescence image and ordinary image is identical to that of the system of the second embodiment. Therefore, it will not be elaborated upon further here and only the operation for imaging a luciferase fluorescence image will be described. Although ultraviolet light cut filter 87 is added to imaging unit 80 of the present embodiment as described above, ultraviolet cut filter 87 is formed of a high-pass filter that passes the ICG fluorescence image and ordinary image, giving no influence on the operation for capturing these images.

When capturing a luciferase fluorescence image, ultraviolet light emitted from the ultraviolet laser light source of light source unit 6 is inputted to optical cable LC3 through condenser lens 71. Then, the ultraviolet light is guided through optical cable LC3 and inputted to body cavity insertion section 30, and further guided through the multimode optical fiber of the ultraviolet light projection unit in body cavity insertion section 30. Then, the ultraviolet light is outputted from the output end of the multimode optical fiber and directed to the observation area.

A luciferase fluorescence image reflected from the observation area irradiated with the ultraviolet light is inputted to insertion member 30b from imaging lens 30d at the tip 30Y of insertion member 30b, then guided by the group of lenses inside of the insertion member 30b, and outputted to imaging unit 80.

The luciferase fluorescence image is reflected in a right angle direction by dichroic prism 81 after passing through ultraviolet light cut filter 87, then formed on the imaging surface of second high sensitivity image sensor 86 by second image forming system 85, and imaged by second high sensitivity image sensor 86 through the blue (B) filters on the imaging surface thereof. Here, ultraviolet light reflected from the observation area is cut by ultraviolet light cut filter 87 and does not enter second high sensitivity image sensor 86.

The luciferase fluorescence image signal outputted from second sensitivity image sensor 86 are subjected to CDS/AGC (correlated double sampling/automatic gain control) and A/D conversion in imaging control unit 80a, and outputted to image processing unit 3 through cable 5.

Note that the imaging timing of the ordinary image, ICG fluorescence image, and luciferase fluorescence image is identical to that of A to C, E, and D of FIG. 12 respectively.

Also note that blue LD light source 40, near infrared LD light source 46, and ultraviolet laser light source in light source unit 6 are drive controlled according to the timing charts of A to E of FIG. 12.

Then, an ordinary image, an ICG fluorescence image, and a composite image are displayed based on the ordinary image signal formed of the R, G, and B signals, ICG fluorescence image signal, and luciferase fluorescence image signal obtained by imaging unit 80 in the manner as described above. The operation of the system for displaying these images is identical to that of the rigid endoscope system of the first embodiment shown in the flowcharts of FIG. 13, 14 except that the luciferase fluorescence image signal is used instead of the fluorescein fluorescence image signal. Therefore, the operation will not be elaborated upon further here.

In the first to third embodiments described above, a blood vessel image is extracted, but images representing other tube portions, such as lymphatic vessels, bile ducts, and the like may also be extracted.

Further, in the first to third embodiments described above, the fluorescence image capturing apparatus of the present invention is applied to a rigid endoscope system, but the apparatus of the present invention may also be applied to other endoscope systems having a soft endoscope. Still further, the fluorescence image capturing apparatus of the present invention is not limited to endoscope applications and may be applied to so-called video camera type medical image capturing systems without an insertion section to be inserted into a body cavity.

What is claimed is:

1. An image obtaining method, comprising the steps of:
obtaining a first image captured by directing light having a first wavelength to an observation area and receiving light emitted from the observation area, and a second image captured by directing light having a second wavelength shorter than the first wavelength to the observation area and receiving light emitted from the observation area; and
obtaining a deep portion image of the observation area by subtracting the second image from the first image.

2. An image obtaining method, comprising the steps of:
obtaining a first fluorescence image captured by directing excitation light having a first wavelength to an observation area and receiving first fluorescence emitted from the observation area, and a second fluorescence image captured by directing excitation light having a second wavelength shorter than the first wavelength to the observation area and receiving second fluorescence emitted from the observation area; and
obtaining a deep portion fluorescence image of the observation area by subtracting the second fluorescence image from the first fluorescence image.

3. An image obtaining method, comprising the steps of:
obtaining a fluorescence image captured by directing excitation light to an observation area and receiving fluorescence emitted from the observation area, and a narrowband image captured by directing narrowband light having a wavelength shorter than that of the excitation light and a bandwidth narrower than that of white light to the observation area and receiving reflection light reflected from the observation area; and
obtaining a deep portion fluorescence image of the observation area by subtracting the narrowband image from the fluorescence image.

4. An image capturing apparatus, comprising:
a light emission unit for emitting first emission light having a first wavelength and second emission light having a second wavelength shorter than the first wavelength, the first and second emission light being directed to an observation area;
an imaging unit for capturing a first image by receiving light emitted from the observation area irradiated with the first emission light and a second image by receiving light emitted from the observation area irradiated with the second emission light; and
a deep portion image obtaining unit for obtaining a deep portion image of the observation area by subtracting the second image from the first image.

5. An image capturing apparatus, comprising:
a light emission unit for emitting first excitation light having a first wavelength and second excitation light having a second wavelength shorter than the first wavelength, the first and second excitation light being directed to an observation area;
an imaging unit for capturing a first fluorescence image by receiving first fluorescence emitted from the observation area irradiated with the first excitation light and a second fluorescence image by receiving second fluorescence emitted from the observation area irradiated with the second excitation light; and
a deep portion image obtaining unit for obtaining a deep portion image of the observation area by subtracting the second fluorescence image from the first fluorescence image.

6. The image capturing apparatus of claim 5, wherein the first excitation light is near infrared light.

7. The image capturing apparatus of claim 5, wherein the light emission unit is a unit that emits the first excitation light and the second excitation light at the same time, and the imaging unit is a unit that captures the first fluorescence image and the second fluorescence image at the same time.

8. An image capturing apparatus, comprising:
a light emission unit for emitting excitation light and narrowband light having a wavelength shorter than that of the excitation light and a bandwidth narrower than that of white light, the excitation light and the narrowband light being directed to an observation area;
an imaging unit for capturing a fluorescence image by receiving fluorescence emitted from the observation area irradiated with the excitation light and a narrowband image by receiving reflection light reflected from the observation area irradiated with the narrowband light; and
a deep portion fluorescence image obtaining unit for obtaining a deep portion fluorescence image of the observation area by subtracting the narrowband image from the fluorescence image.

9. The image capturing apparatus of claim 8, wherein the excitation light is near infrared light.

10. The image capturing apparatus of claim 8, wherein the light emission unit is a unit that emits the excitation light and the narrowband light at the same time, and the imaging unit is a unit that captures the fluorescence image and the narrowband image at the same time.
